(19) United States
(12) Patent Application Publication
(10) Pub. No.: US 2005/0216008 A1
(43) Pub. Date: Sep. 29, 2005

(54) Bone fixation implants

(76) Inventors: Ralph Fritz Zwirnmann, Roslyn, PA (US); Douglas Howard Vaughen, Downingtown, PA (US); Michael Chen, Philadelphia, PA (US)

Correspondence Address: MORGAN, LEWIS & BOCKIUS LLP, 1701 MARKET STREET, PHILADELPHIA, PA, 19103-2921, US

(21) Appl. No.: 10/809,768

(22) Filed: Mar. 24, 2004

(51) Int. Cl. A61B 17/80

(52) U.S. Cl. 606/69; 606/70

(57) ABSTRACT

The present invention is generally directed to an improved bone fixation implant that provides tactile indication of the top surface of the implant. In one embodiment, an implant comprises a top surface, a bottom bone-contacting surface, and recessed portion disposed in the top surface of the implant to provide a tactile indicator for readily identifying the top surface of the implant. In another embodiment, the top surface indicator may be in the form of an elongated top surface groove that is recessed below the top surface of the implant. The implant may further include at least two fastener holes for receiving fasteners therethrough to secure the implant to the bone. In preferred embodiments, the implant may be made of a resorbable or metallic materials. In another embodiment of the present invention, an implant may comprise transverse slots preferably disposed between at least some of the fastener holes.

### FIELD OF THE INVENTION

The present invention generally relates to implants for bone fixation such as plates and meshes, and more particularly to improved implants having for example an indicator that permits the top surface of the implant to be more easily detected during surgery.

### BACKGROUND OF THE INVENTION

Biologically compatible metallic and resorbable implants, such as differently shaped plates and meshes, have been used in craniofacial surgical bone repair applications. Such implants are used to mend bone discontinuities resulting from trauma-induced fractures or osteotomies necessitated by various surgical procedures. These implants are commonly secured to the bone with various types and shapes of fasteners, such as screws and tacks. Craniofacial plating has been offered in a variety of configurations including plates with straight-sides (as shown in FIG. 1) or undulating sides being wider at the fastener holes than between the holes (as shown in FIG. 2). The undulating plate design provides desirable bending characteristics ensuring that such plates bend between the fastener holes (typically the weakest part of the plate) and allows such plates to be readily contoured to match the shape of the bone to which they will be secured.

Unlike metallic bone implants which have been commonly used, resorbable plates offer many desirable properties that are particularly well adapted for certain surgical applications, such as those involving craniofacial bone repair. For instance, resorbable plates retain their necessary strength for a predetermined period of time following implantation to allow the bone discontinuity (resulting from a traumatic fracture or intentional incision made for other surgical purposes) to mend. After the implant has served its useful structural purpose and preferably when the bone has satisfactorily mended, these resorbable implants dissolve and are absorbed by the patient's body through natural mechanisms such as hydrolysis. This is especially advantageous for patients such as children and young adults where bone development and growth is still occurring. In these young patients, resorbable implants may be indicated to avoid some potential complications associated with metallic implants which are not absorbed by the body and may impede normal bone development or migrate from the original location if not removed by a second surgery.

Resorbable implants may be made from various materials, including polymers and combinations of two or more polymers to create copolymers, terpolymers, etc. The selection of individual and combinations of various polymers, methods used to manufacture the polymers and bone implants themselves, and other factors may affect the functional properties of the resorbable implants, such as how long structural strength and dimensional stability is retained after implantation and the time required for complete absorption.

Resorbable materials are generally relatively rigid and inflexible at ambient operating room and human body temperatures. As inherent with many polymers, resorbable materials become more flexible and bendable when their temperature is elevated to a temperature above the glass transition temperature (T_{g}) of the material. Accordingly, resorbable implants may be bent to match the contour of the bone surface to which they will be attached by first heating the implant to a temperature above the glass transition temperature (T_{g}) and below the melting point of the material by means such as a water bath, heating wand, hot air blower, or other suitable method known in the art. When the temperature of the implant is allowed to fall back below the glass transition temperature (T_{g}) of the material, the implant will return to its initial substantially rigid condition and hold the shape into which it has been formed.

Unlike much larger metallic implants commonly used for orthopedic fracture fixation of long bones such as the femur and humerus, both resorbable and metallic bone implants used in craniofacial applications are significantly shorter in length, narrower, and thinner. They are sometimes referred to as mini plates in the art. For example, some craniofacial plates may typically be less than 1 inch in length, about ¼ inch or less in width, and less than ⅛ inch in thickness. In addition, resorbable implants (e.g., plates and meshes) are relatively translucent and semi-transparent in appearance which may make it difficult for some surgeons to readily distinguish the top and bottom surfaces of the implant. Thus, the size of these craniofacial implants and/or the type of material used to make the implants (e.g., resorbable polymers) can make it cumbersome for some surgeons to quickly identify the top surface of the implant during surgery. This is especially important for proper installation where the fastener holes do not have a constant diameter between the upper and lower surfaces of the implant, but may for example have countersunk holes intended to receive fasteners having heads of a corresponding shape. Improvements in packaging and labeling implants have attempted to solve this problem, but these measures have had only limited success.

Thus, there is a need for bone implants that can provide the surgeon with direct and positive indication of the top surface of the implant to facilitate proper surgical installation of the implant onto the bone.

### SUMMARY OF THE INVENTION

The invention relates to a bone implant that is configured and dimensioned to provide positive indication of the top surface of the implant via tactile means. Implants according to principles of the present invention generally comprise a top surface, a bottom bone-contacting surface, and at least one recessed region or portion disposed in the top surface of the implant to provide a tactile indicator for readily identifying the top surface of the implant. In one embodiment, the top surface indicator may be in the form of an elongated groove that is recessed below the top surface of the implant. Preferably, the top surface groove may be U-shaped in cross-section; however, other suitable cross-sectional shapes are contemplated and may be used as a matter of design choice. Also preferably, the top surface groove extends only partially through the thickness of the implant to distinguish the top surface from the bottom surface. In one embodiment, the implant is made from a resorbable material.

In one embodiment, the implant has at least one elongate plate section. In other embodiments, the implant incorporates at least one or more elongate plate sections and has a form which may be an L-shape, a Y-shape, a double Y-shape, an X-shape, or other numerous shapes which may be formed by combining various elongate plate sections.

It will be appreciated by one skilled in the art that depending on the shape and size of the implant, a plurality of recessed top surface portions may be provided and arranged in numerous layout patterns when looking at the plate in plan view from above. In one embodiment, for example, a mesh plate implant may have a plurality of crossing elongated recessed portions arranged diagonally, parallel, or in a combination thereof to the sides of the mesh plate. In addition, the recessed portion itself may be provided in any number of cross-sectional shapes and combinations of shapes thereof without limitation. In one preferred embodiment, for example, the recessed top surface groove may have a U-shaped cross-sectional shape in the form of a square or rectangular channel.

The implant may further include at least one fastener hole extending from the top surface to the bottom surface of the implant. A variety of screws, tacks, or similar fasteners may be installed through the holes to affix the implant to the bone. Preferably, at least two holes are provided. The fastener holes may have straight walls forming a constant diameter hole from the top to bottom surface of the implant. Preferably, the top surface of the implant has countersunk regions around the fastener holes such that the heads of fasteners inserted therethrough may be substantially flush with the top of the implant.

The implant may also have chamfered side edges to provide additional tactile indication of the top surface of the plate. These chamfered edges also allow the implant to be less palpable after implantation.

It will be appreciated by one skilled in the art that providing a recessed portion in the top surface of an implant, such as a longitudinal groove discussed above, may alter the implant flexural rigidity and out-of-plane bending properties because the section modulus of the implant is affected by its cross-sectional dimensions and shape. The term “out-of-plane” is defined as the direction normal to the top or bottom surfaces of the implant. In addition, in the case of some prior elongate bone plate designs having undulating sides such as that shown in FIG. 2, it may be necessary to concomitantly widen the plate between the fastener holes to physically accommodate an elongated top surface groove or channel as described above. Thus, according to another aspect of the present invention, it has been determined that advantageously adding transverse openings (preferably such as slots or slits, for example) partially across the width of the bone plate may be used as a means to adjust or control the out-of-plane bending characteristics of the plate. Accordingly, where an elongated top surface groove is added to an existing plate design to provide top surface indication accordingly to principles of the present invention, transverse openings may be used in conjunction with the surface groove if the designer wishes to maintain comparable out-of-plane bending characteristics. Alternatively, it will be appreciated that transverse openings may also be used alone with equal benefit in bone plates not having an elongate surface indication groove as a means to regulate the bending and flexural rigidity characteristics of the plate. Accordingly, the use of transverse openings is not limited to use with top surface indication recesses.

In light of the foregoing, therefore, a bone plate formed according to another aspect of the present invention may also comprise a top surface, a bottom surface, a plurality of fastener holes, a recessed portion in the top surface of the plate, and at least one transverse opening running across the width of the plate. The bone plate may comprise at least one elongate plate section. The recessed portion is preferably an elongate groove; however, other shapes are contemplated. In one embodiment, the elongate groove extends at least partially between two fastener holes. The transverse opening may be elongate, and in the form of a slit or slot, for example. In some embodiments, the transverse slots may be preferably oriented perpendicular to the longitudinal axis of the plate. Preferably, the transverse openings may be disposed between at least some of the fastener holes. Also preferably, the transverse openings may extend both at least partially across the width of the plate and partially through the thickness of the plate. More preferably, the transverse openings extend all the way through the plate from the top surface to the bottom surface. In preferred bone plate embodiments having a plurality of fastener holes, the transverse openings may be provided between at least some of the fastener holes. In another embodiment, a bone plate with bending control includes at least one elongate tranverse slot as described above, but does not include a tactile top surface indicator in the form of an elongate groove or recessed region. Bone plates with bending control may be made from any suitable biocompatible material as described herein, including a resorbable material.

It should be recognized that the transverse openings also advantageously promote elongate plates to bend between, and not at the fastener holes. The fastener holes are typically the weakest part of the plate and experience the highest stresses caused by external loads imposed on the plate after implantation. Thus, it is preferable that such plates bend between the fastener holes to reduce the likelihood of failure.

In another embodiment, a bone plate with bending control formed according to principles of the present invention comprises a top surface and a bottom bone-contacting surface, at least two fastener holes defining a longitudinal axis therebetween and disposed in the plate extending from the top surface to the bottom surface of the plate, and at least one elongate slot disposed in the plate and extending from the top surface to the bottom surface of the plate. The elongate slot preferably may be disposed between the two fastener openings and extends transverse to the longitudinal axis. The elongate transverse slot affects the bending characteristics of the plate and induces the plate to bend between the fastener holes.

A method of contouring and attaching resorbable implants having top surface indicators to the bone is also provided. The method comprises the steps of: providing a resorbable implant having a glass transition temperature (T_{g}) that is higher than average human body temperature, the implant comprising a top surface and a bottom bone-contacting surface, at least two fastener holes extending from the top surface to the bottom surface, and a portion of the top surface being recessed and extending partially between the top and bottom surfaces, whereby the top surface recess provides a tactile indicator for identifying the top surface of the implant; raising the temperature of the implant to above the glass transition temperature (T_{g}); touching the surfaces of the plate to find the top surface recess thereby identifying the top surface; deforming the plate to substantially conform to the anatomical shape of the bone with the top surface facing away from the bone; applying the plate to the bone; and attaching the plate to the bone. The method may further include providing fasteners and inserting the fasteners through at least some of the fastener holes, wherein the fasteners are used for attaching the plate to the bone. In one embodiment, the fasteners are screws or tacks.

A bone fixation kit as described hereafter is also provided. In general, the kit may comprise at least a first bone implant and fasteners for securing the implant to a bone. Preferably, the implant may have a recessed region in the top surface to provide a tactile indicator for identifying the top surface of the implant. The implant and/or fasteners may be made from a resorbable material. In other embodiments, the kit may include at least a second and at least a third bone implants. Accordingly, the kit may include without limitation a combination of any number, sizes, design, and/or shapes of bone implants and fasteners as described herein.

It will further be appreciated by one skilled in the art that the invention is particularly useful for craniofacial skeleton surgical implants, including such implants that are made of biologically compatible metals (stainless steel, titanium, etc.), resorbable materials, composite materials, and other suitable implant materials known in the art. Preferably, implants formed according to principles of the present inventions may be made from resorbable materials, discussed in more detail below.

It should be noted that use of the invention is not limited to craniofacial applications, nor is the manufacture of the invention limited to the foregoing materials. Accordingly, the invention may be used for any type of implant where it is desirable to provide a positive tactile indication of the top surface of the implant and/or control the bending characteristics of the implant.

### BRIEF DESCRIPTION OF THE DRAWINGS

The features and advantages of the present invention will become more readily apparent from the following detailed description of the invention in which like elements are labeled similarly, and in which:

FIG. 1 is a top plan view of a straight prior art bone plate;

FIG. 2 is a top plan view of an undulating prior art bone plate;

FIG. 3A is a top plan view of an implant in the form of a bone plate formed according to principles of the present invention having a top surface indicator;

FIG. 3B is a cross-sectional view taken along line 3B-3B in FIG. 3A;

FIG. 3C is a cross-sectional view taken through a fastener hole along line 3C-3C in FIG. 3A;

FIG. 3D is a perspective view of the bone plate of FIG. 3A;

FIG. 3E is a cross-sectional view of the bone plate of FIG. 3A having an alternative V-shaped embodiment of a top surface indicator;

FIG. 3F is a cross-sectional view of the bone plate of FIG. 3A having an alternative concave-shaped embodiment of a top surface indicator;

FIG. 4 is a side view of a screw useable with the implant of FIG. 3A having a head configured to mate with the fastener holes of the implant of FIG. 3A;

FIG. 5 is a top plan view of a different embodiment of a bone plate according to the present invention having four fastener holes;

FIG. 6A is a top plan view of another embodiment of a bone plate according to principles of the present invention having a top surface indicator, transverse slots, and a plurality of fastener holes;

FIG. 6B is a cross-sectional view taken through a fastener hole along line 6B-6B in FIG. 6A;

FIG. 7 is a top plan view of another embodiment of an implant formed according to principles of the present invention in the form a strut plate having top surface indicators and transverse slots;

FIG. 8 is a top plan view of another embodiment of an implant formed according to principles of the present invention in the form of an orbital rim plate having a top surface indicator and transverse slots;

FIG. 9 is a top plan view of another embodiment of an implant formed according to principles of the present invention in the form of a left L-plate having a top surface indicator and transverse slots;

FIG. 10 is a top plan view of another embodiment of an implant formed according to principles of the present invention in the form of a right L-plate having a top surface indicator and transverse slots;

FIG. 11 is a top plan view of another embodiment of an implant formed according to principles of the present invention in the form of a Y-plate having a top surface indicator and transverse slots;

FIG. 12 is a top plan view of another embodiment of an implant formed according to principles of the present invention in the form of a double Y-plate having a top surface indicator and transverse slots;

FIG. 13 is a top plan view of another embodiment of an implant formed according to principles of the present invention in the form of an X-plate having a top surface indicator and transverse slots;

FIG. 14 is a top plan view of another embodiment of an implant formed according to principles of the present invention in the form of a burr hole cover plate having a top surface indicator and transverse slots;

FIG. 15A is a top plan view of another embodiment of an implant formed according to principles of the present invention in the form of a resorbable mesh plate having a top surface indicator;

FIG. 15B is a cross-sectional view taken through a fastener hole along line 15B-15B in FIG. 15A having a top surface indicator;

FIG. 16 is a side view of a tack useable with all implants similar to the one depicted in FIG. 3A having a head configured to mate with the fastener holes of the implant of FIG. 3A; and

FIGS. 17A-Q depict several different components that may, in any number of combinations, compose a bone implant kit with surface indicator.

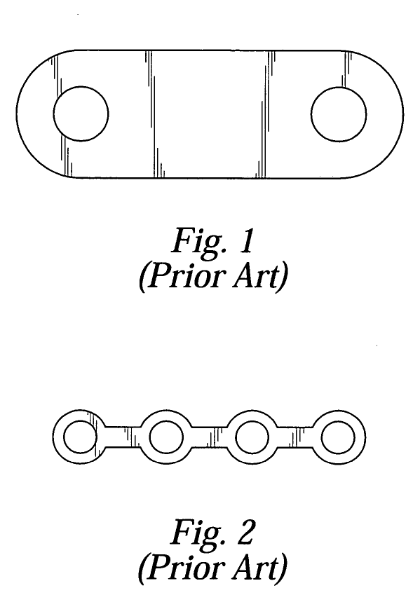

### DESCRIPTION OF THE PREFERRED EMBODIMENTS

In the description that follows, any reference to direction or orientation is merely intended for convenience of description and is not intended in any way to limit the scope of the present invention. Moreover, the features and benefits of the invention are illustrated by reference to the preferred embodiments. Accordingly, the invention expressly should not be limited to such preferred embodiments illustrating some possible non-limiting combination of features that may exist in alone or in other combinations of features, and which should only be limited by the claims appended hereto.

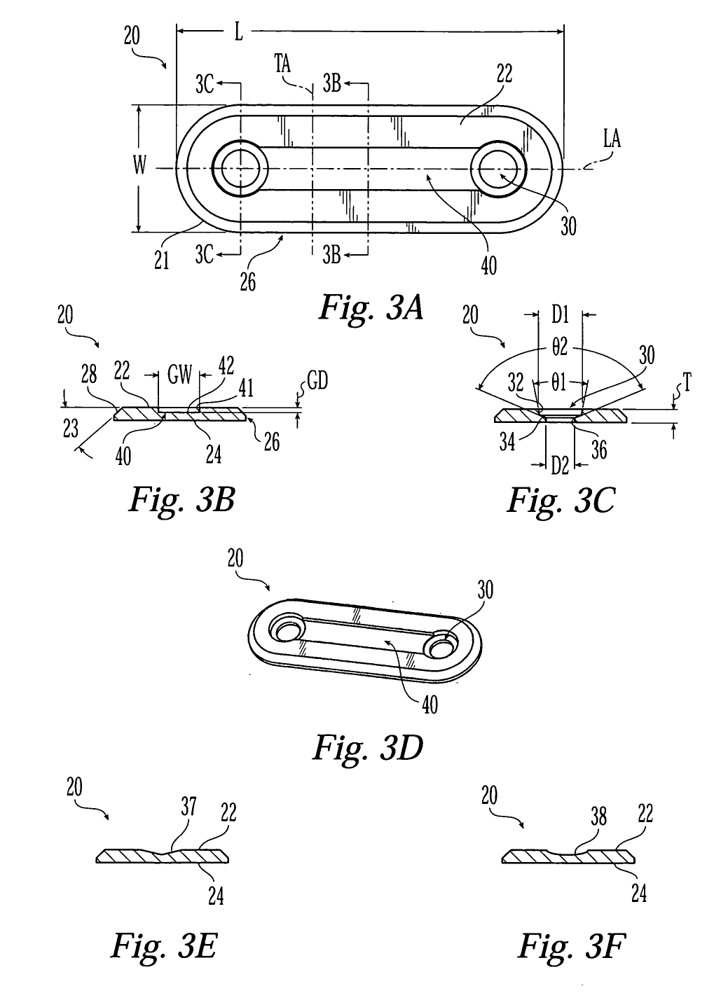

FIGS. 3A-D depict one embodiment of an implant for bone fixation in the form of a bone plate. The bone plate 20 has a generally elongate body defining a longitudinal axis LA extending along the centerline of the plate, and a transverse axis TA extending perpendicular to the longitudinal axis. Plate 20 includes two ends 21, a top surface 22, a bottom bone-contacting surface 24, two longitudinally extending sides 26 connecting the top to bottom surfaces, and two ends 21. Preferably, ends 21 are rounded in shape (as shown) to avoid possible soft tissue irritation, but ends 21 may have any suitable configuration. The distance between the top surface 22 and bottom surface of plate 20 defines a thickness T for the plate. Preferably, thickness T is substantially constant from one end of plate 20 to the other end, but it may vary along the longitudinal axis LA, the transverse axis TA, or both.

Preferably, at least two fastener holes 30 are provided in plate 20 which may be located near the ends 21 of the plate. Holes 30 extend from the top surface 22 to the bottom surface 24 and are configured to receive a fastener to attach plate 20 to the bone. FIG. 3C, a cross-sectional view taken from FIG. 3A, shows one preferred embodiment of a countersunk fastener hole 30. Starting at the top surface of plate 20, hole 30 preferably has a conical countersunk shape comprising a first inclined wall 32, followed by an adjacent second inclined wall 34, and followed again by a straight-wall that penetrates the bottom surface 24 of plate 20. First inclined wall 32 of hole 30 has a different angle θ1 than second inclined wall 34 which has an angle θ2. Preferably, θ1 is about 15-25 degrees, preferably about 20 degrees, and θ2 is about 130-150 degrees, preferably about 140 degrees.

It should be noted that the number of fastener holes 30 provided are typically dictated by the length of the bone plate and the number of possible fastener mounting locations intended to be provided, both being generally a matter of design discretion.

It should be noted that hole 30 may be of any suitable shape and is not limited to the shape described above. For example, hole 30 may be conical countersunk in shape with only a single inclined wall, or hole 30 may have entirely straight walls without any countersunk portion, or hole 30 may be be spherical in cross-sectional shape. Accordingly, the present invention is not limited by the shape of hole 30.

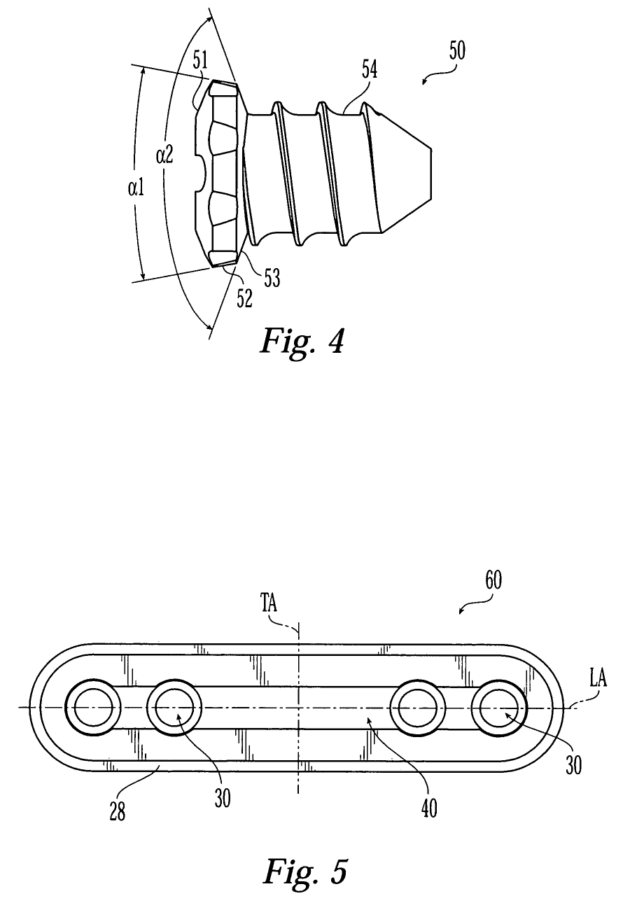

The conical countersunk hole 30, as shown in FIG. 3C, may be preferably used with fasteners having a matching fastener head configuration with double-inclined walls. For example, screw 50 (shown in FIG. 4) may be used in hole 30 and has a threaded shank 54 and head 51 with inclined surfaces 52, 53 corresponding in shape to inclined walls 32, 34 of hole 30, respectively. Accordingly, screw head 51 inclined surfaces 52, 53 preferably have angles α1 and α2 to match angles θ1 and θ2 of hole 30, respectively. The top of screw head 51 may be slightly convex, as shown. An advantage of the mating screw and fastener hole configurations is that the top of the screw is substantially flush with the top surface 22 of plate 20 when the screw 50 is installed in countersunk hole 30 (except for the slight convexity of the top of the screw head 51). This helps reduce possible soft tissue irritation when plate 20 is implanted, and the screw heads 51 cannot readily and separately be felt beneath the skin by the patient, particularly in locations where there is a relatively thin skin coverage over the bone, such as in craniofacial applications.

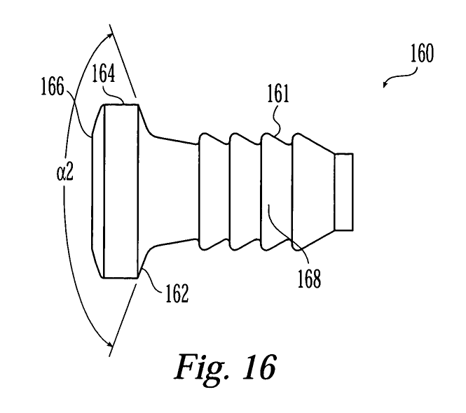

In another embodiment shown in FIG. 16, a fastener in the form of a tack 160 may also used in fastener hole 30 of plate 20. Tack 160 has a head 166 and corrugated shank 168 with corrugations 162 running substantially transverse to the longitudinal axis of the tack. Tack head 166 may have a straight side surface 164 and an inclined surface 162 which forms an angled transition to shank 168. At least a portion of inclined surface 162 has an angle α2 that cooperates with angle θ2 of hole 30, thereby matching the shape of inclined wall 34 of hole 30 in a similar fashion to screw 50 discussed above. Tack 160 is used by the surgeon drilling a hole in the bone that is slightly smaller in diameter than the outermost diameter of corrugations 162. Tack 160 is then pressed into the bone hole and held in place by a friction fit.

Referring again to FIGS. 3A-F, plate 20 further comprises a recessed surface region or portion that serves as a top surface indicator. Preferably, the recessed region is separate from the fastener holes; however, the recessed region may intersect and incorporate one or more fastener holes. In one embodiment, the top surface indicator is a longitudinally extending elongate groove 40 that is recessed below the top surface 22 of plate 20. Preferably, top surface groove 40 extends at least partially along the length L of plate 20, partially across the width W of plate 20, and preferably partially through the thickness T of plate 20 to distinguish the top surface 22 from the bottom surface 24. Also preferably, top surface groove 40 extends between the fastener holes and more preferably, completely from one hole 30 to the other hole.

Top surface groove 40 provides a tactile indicator making it easier for a surgeon to identify the top surface 22 of plate 20 by touch. This helps to ensure that the plate 20 is properly attached to the bone such that the countersunk fastener holes 30 are facing upwards for receiving bone screws 50 or tacks 160. Although top surface groove 40 is particularly useful with implants such as plate 20 described above where the fastener holes are not symmetrical at the top and bottom surfaces of the implant, it will be appreciated that top surface groove 40 is not limited to such applications and may be used with any implants where it is desirable to identify the top surface of the implant.

In one preferred embodiment, top surface groove 40 may be U-shaped in cross section (best seen in FIG. 3B) having a substantially planar bottom surface 42 and planar sidewalls 41. Preferably, the depth GD and width GW of top surface groove 40 is sufficient to allow a surgeon or other individual to easily identify the top surface 22 of the plate by tactile touch during a procedure; preferably, while the surgeon or other individual may be wearing latex gloves. Also preferably, the bottom surface 24 of plate 20 may have a relatively smooth profile to facilitate differentiating the opposite grooved top surface 22 of plate 20 from the bottom.

Although top surface groove 40 may preferably be channel shaped, it will be appreciated that top surface groove 40 may have various other suitable cross-sectional configurations, such as but not limited to a V-shaped groove 37 (see FIG. 3E), concave-shaped groove 38 (see FIG. 3F), etc. Thus, other shaped grooves are contemplated. In addition, it will be appreciated that depth GD and width GW of top surface groove 40 may be varied as a matter of design choice. Accordingly, the invention is not limited with respect to either the shape or size of top surface groove 40. Also preferably, elongated top surface groove 40 is disposed along the centerline of plate 20 which coincides with the longitudinal axis LA, as shown in FIG. 3A. However, the location of groove 40 is not limited in this regard and other locations are contemplated.

Referring to FIG. 3B, at least a portion of the sides 26 and/or ends 21 of plate 20 may have an edge chamfer 28 to provide an additional tactile indicator for identifying the top surface 22 of the plate. The chamfered edge also allows the implant to be less palpable after implantation. Preferably, the chamfer 28 extends completely around plate 20. Also preferably, the chamfer has an angle 23 of about 40-50 degrees, more preferably about 45 degrees, to the top surface 22 of plate 20.

In one embodiment of a straight elongate plate, the elongate portion of the plate (see, e.g., FIGS. 3A, 5, and 6A) may typically be approximately 0.8 mm thick T by 6 mm wide W and have a top surface groove 40 typically measuring about 2.0 mm wide GW by 0.3 mm deep GD (dimensions±allowances for manufacturing tolerances). In another embodiment of a straight elongate plate, the elongate portion of the plate for receiving 2.0 mm nominal diameter bone fasteners may typically be approximately 1.2 mm thick by 7 mm wide with about a 2.0 mm wide by 0.3 mm deep top surface groove 40. The length L of the bone plates may be varied as a matter of design choice and the specific anatomical skeletal portions intended for the plates.

FIG. 5 depicts another embodiment of the present invention comprising a bone plate similar to plate 20 as shown in FIGS. 3A-D, but with four fastener holes 30 instead of two. Plate 60 may be longer than plate 20, and the additional fastener holes provides extra mounting flexibility and/or security in attachment to the bone. Preferably, top surface groove 40 extends at least between the innermost two fastener holes 30. More preferably, top surface groove 40 connects all four fastener holes 30.

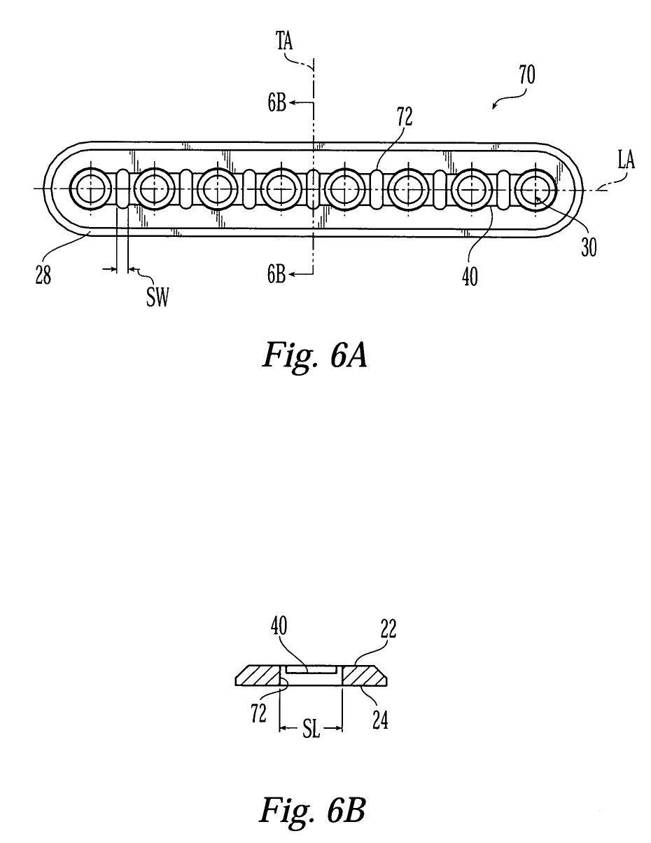

Referring now to FIG. 6A-C, there is shown another embodiment of a bone plate according to the present invention incorporating transverse openings which may be in the form of slits or slots, as discussed above. The number, size, and location of the transverse slots or slits allow the designer to control the bending characteristics of the plate. A straight elongate bone plate 70 incorporating transverse slits or slots may comprise a plurality of fastener holes 30 (see FIG. 3C), and preferably a longitudinally extending top surface groove 40 that extends between the fastener holes. Generally elongate transverse slots 72 extend preferably transverse to the longitudinal axis LA of plate 70 and preferably between the fastener holes 30. Preferably, transverse slot 72 intersects top surface groove 40 and preferably extends from the top surface 22 to the bottom bone-contacting surface 24 of plate 70. However, slot 72 alternatively may extend only partially between the top surface 22 to the bottom bone-contacting surface 24 of plate 70. Also preferably, a plurality of elongate transverse slots 40 are provided and evenly spaced between at least some of the fastener holes 30.

Although FIGS. 6A-C depict a relatively close fastener hole 30 arrangement with one transverse slot 72 disposed between each pair of holes 30, the invention is not limited in this regard. Accordingly, any number and spacing of fastener holes 30 may be used with any number and spacing of transverse slots 72, all being a matter of design choice. For example, elongate plate 20 shown in FIG. 3A may include transverse slots 72. In addition, transverse slits or slots 72 may be provided for elongate implants that do not have a top surface indicator such as elongate top surface groove 40. Moreover, more than one transverse slot 70, which may be of different configuration and orientation, may be provided between fastener holes 30.

As discussed above, transverse slots 40 promotes elongate plates such as plate 70 to bend between, and not at the fastener holes 30 which typically are the weakest points in the plate and experience the highest bending stresses. In addition, transverse slots 40 allow the designer to control the bending characteristics of the plate and the plate's flexural rigidity. Also as noted above, it will be understood that altering the dimensions, shape, and number of slots 72 provides the designer with a means to alter the bending characteristics of the plate.

With reference to FIGS. 6A and 6B, transverse slots 40 in one embodiment may typically measure about 0.8 mm in width SW by about 2.5 mm in length SL.

The implants of the present invention may be made from any biocompatible material, including, but not limited to metals, resorbables, composites (i.e., combinations of various materials either in an integrated or laminate construction), etc. As discussed above, implants of the present invention may preferably be made from any suitable resorbable (i.e., biodegradable and bioabsorbable) material. These materials eventually dissolve over time following implantation and are absorbed by the patient's body. More preferably, the implants may be made from polymer-based resorbables including, but are not limited to, one type of polymer, combinations of two or more different polymers to create various copolymers, terpolymers, etc., polymer alloys, composites having multiple layers of resorbable polymers, polymers containing resorbable reinforcement fibers, etc. The selection of material and individual or combinations of various polymers, methods used to manufacture the polymers and implants, and other factors affect the functional properties of the resorbable implants, such as how long structural strength and dimensional stability is retained in vivo after implantation and the time required for complete absorption of the implant by the patient's body.

Resorbable polymeric materials are generally somewhat rigid and inflexible at ambient operating room and human body temperatures. Such polymers typically become more flexible and bendable when their temperature is elevated to a temperature above the glass transition temperature (T_{g}) and below the melting point of the material. Accordingly, resorbable implants may be bent to match the three-dimensional contour of the bone surface to which they will be attached by heating the implant to a temperature above the glass transition temperature (T_{g}) and below the melting point of the material by means such as a water bath, hot air gun, in situ bending/cutting iron, or other suitable means known in the art. Once the resorbable implant has been contoured and secured in place on the bone, rigidity returns as its temperature drops below the glass transition temperature (T_{g}).

Preferably, an implant formed according to principles of the present invention may be made from polymers such as lactide and glycolide, and copolymers of the same. More preferably, the implant is made of 70/30 poly (L, D/L-lactide) copolymer or 85/15 poly (L-lactide-co-glycolide) copolymer compositions. These compositions have desirable mechanical and resorption properties, such as sufficiently long in vivo strength retention after implantation to allow sufficient time for bone mending to occur.

It will be appreciated that processing of the raw polymeric material(s) and manufacturing methods can effect the properties of the polymers and implants.

Preferably, an implant made from the 70/30 poly (L, D/L-lactide) copolymer composition may be fully resorbed within approximately 3 years±after being implanted. An implant made from the 85/15 poly (L-lactide-co-glycolide) copolymer composition may preferably be fully resorbed within approximately 1 year±after being implanted. It will be appreciated that the thickness of the implant, its geometric configuration, and individual patient's body chemistry may affect the resorption times.

Implants formed according to principles of the present invention may be made from polymers that are crystalline or amorphous (i.e., non-crystalline) in structure, depending on the specific raw polymeric material(s) selected to fabricate the implant, processing of the raw polymeric material(s), and method used to manufacture the finished implant, all of which are a matter of design choice. Thus, the crystallinity of the polymer raw material and finished implant may be varied as a matter of design choice. In one embodiment, the polymer raw material of the 70/30 poly (L, D/L-lactide) copolymer composition (i.e., before the implant is formed) has a raw material crystallinity preferably of approximately 10-12%. In another embodiment, the copolymer raw material of the 85/15 poly (L-lactide-co-glycolide) copolymer composition (i.e., before the implant is formed) preferably has a crystallinity of approximately 15-35%.

The materials and implants according to principles of the invention may also contain or be impregnated with various additives, fillers, chemical and biologically-active agents (i.e., antibiotics, pharmaceuticals, proteins, etc.), surface treatments, etc. to alter and/or facilitate the processing, manufacture, properties, and/or performance of the materials and implants. The implant may further be coated with materials that contain or are biologically active agents, antibiotics, medicinals, growth factors, etc.

Resorbable polymeric implants made according to principles of the present invention are preferably compression molded in one embodiment. Preferably, fasteners used to secure implants of the present invention to the bone are also made from resorbable materials, preferably the same polymeric resorbable material from which the implants are made. The fasteners, however, may also be made from different resorbable materials than the implants. Preferably, the fasteners may be injection molded.

Implants made from the foregoing 70/30 poly (L, D/L-lactide) copolymer and 85/15 poly (L-lactide-co-glycolide) copolymer compositions preferably have a glass transition temperature (T_{g}) that is above ambient operating room and human body temperatures. In one embodiment, the glass transition temperature Tg is at least about 50 degrees C. As noted above, resorbable polymers are generally somewhat rigid and inflexible below the glass transition temperature Tg. When heated to temperatures above the glass transition temperature Tg and below the melting point of the material, the resorbable polymers become more flexible and may readily be bent by the surgeon to conform to the anatomical shape of the bone intended to receive the implant.

Implants of the present invention are preferably made, without limitation, by cutting the implants from a compression molded plain sheet of resorbable material. In one embodiment, the plain sheet may typically measure 150 mm square. A single sheet may yield more than one implant or plate, and the top of the sheet may become the top surface of the finished implants or plates. All features of the plates are preferably similarly cut or machined into the implants at the factory, including top surface grooves, fastener holes, edge chamfers, transverse slots, etc.

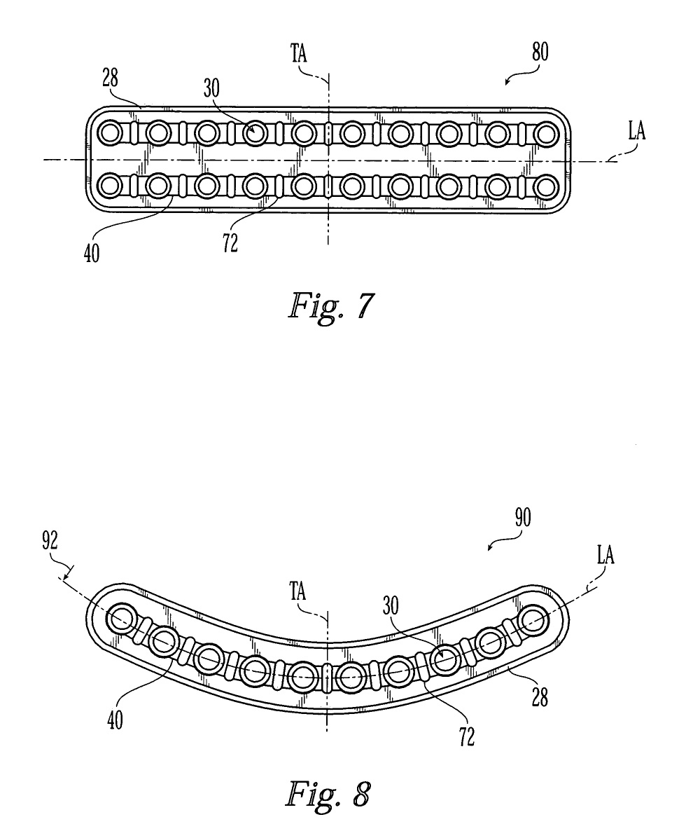

Implants of the present invention are not limited in shape to the generally elongate straight bone plates discussed above, which are used merely for convenience to describe some possible illustrative and non-limiting preferred embodiments of the invention. Thus, numerous other implant configurations are possible that may be formed according to the principles of the present invention. For example, as shown in FIGS. 7-16, other possible shapes without limitation are double-wide broadened strut plates, curved orbital rim plates, L-plates, Y-plates, double Y-plates, X-plates, burr hole covers, box plates and meshes. Some of these implants may comprise portions or sections of two or more individual generally elongate straight plates described heretofore that are combined to create various other configurations.

Other possible embodiments of implant shapes according to principles of the present invention will now be briefly described.

FIG. 7 shows another embodiment of the present invention in the form of double-wide strut plate. Generally elongate plate 80 is similar to plate 70 shown in FIG. 6A; however, plate 80 has a double row of both holes 30 and top surface grooves 40. Plate 80 preferably includes countersunk holes 30, elongate top surface grooves 40 extending between at least some of the holes, transverse slots 72, and side edge chamfer 28.

FIG. 8 shows another embodiment of the present invention in the form of a curved orbital rim plate. Generally elongate plate 90 is similar to plate 70 shown in FIG. 6A; however, plate 90 has a slightly curved arc-like shape with a radius 92 to match the average shape of the orbital rim for use in reconstructive surgery of the bone involving the eye socket. In one embodiment, radius 92 is preferably about 32 mm, but may vary infinitely to match patient anatomy. Plate 90 also preferably includes countersunk holes 30, elongate top surface groove 40 extending between at least some of the holes, transverse slots 72, and side edge chamfer 28.

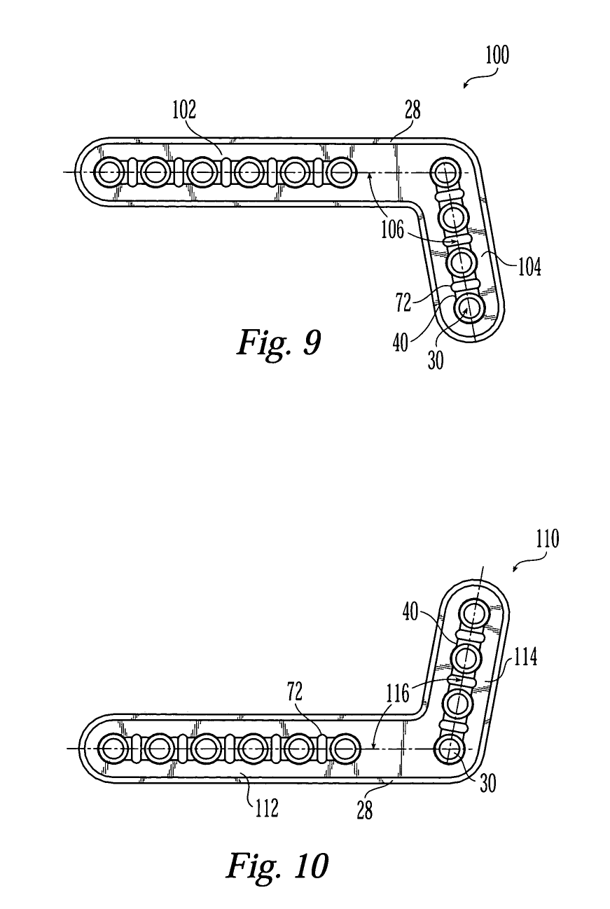

FIG. 9 shows another embodiment of the present invention in the form of an L-plate. Generally plate 100 comprises two elongate plate sections each similar to the plate 70 shown in FIG. 6A; however, one plate is disposed at an angle to the other plate. Plate 100 has an elongate body portion 102 and an elongate head portion 104 disposed at an angle 106 to the body portion. Preferably, angle 106 is at least 90 degrees, but may be any angle greater or less than 90 degrees. More preferably, angle 106 is an oblique angle greater than 90 degrees. In one preferred embodiment, angle 106 is about 100-110 degrees. In the embodiment shown, body portion 102 is preferably longer than head portion 104. Both body portion 102 and head portion 104 also each preferably include countersunk holes 30, elongate top surface groove 40 extending between at least some of the holes, transverse slots 72, and side edge chamfer 28. It should be noted that plate 100 of FIG. 9A may conveniently be referred to as a left oblique L-plate.

FIG. 10 shows another embodiment of the present invention in the form of a right oblique L-plate. Plate 110 is similar to left oblique L-plate 100, but is generally a mirror image of plate 100. Accordingly, right oblique L-plate 110 has an elongate body portion 112 and an elongate head portion 114 disposed at an angle 116 to the body portion. The other features of L-plate 110 (i.e., fastener holes 30, elongate top surface groove 40, transverse slots 72, side edge chamfer 28) are essentially the same as in L-plate 100.

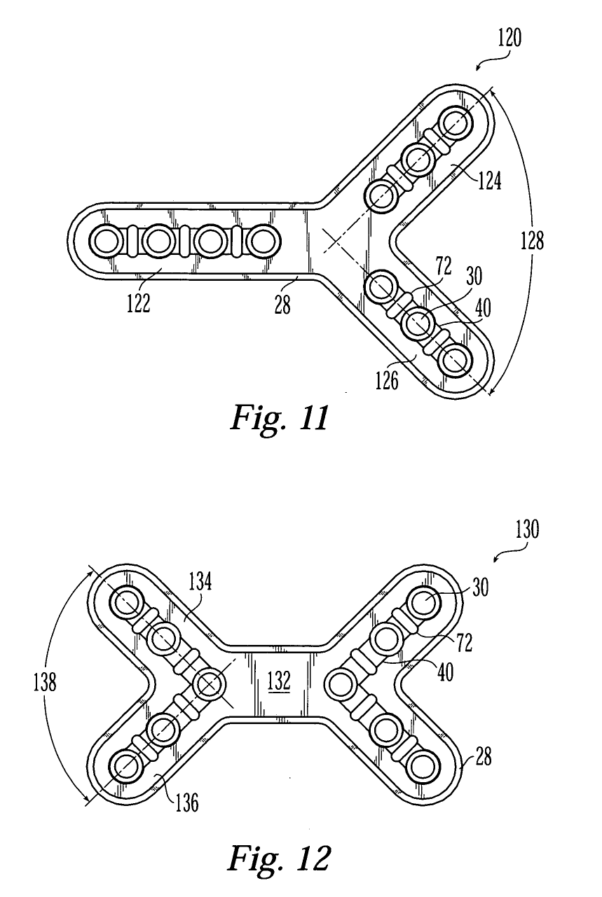

FIG. 11 shows another embodiment of the present invention in the form of a Y-plate. Generally plate 120 comprises three elongate plate sections each similar to plate 70 and combined in the manner shown in FIG. 11. Plate 120 has an elongate body portion 122 and an elongate first head portion 124 and an elongate second head portion 126. Angle 128 is formed between first and second head portions 124, 126, respectively. Preferably, angle 128 is less than or equal to about 180 degrees. More preferably, angle 128 is about 70-110 degrees, and even more preferably about 90 degrees. In the embodiment shown, body portion 122 is preferably longer than either first head portion 124 or second head portion 126, but is not limited in its length with respect to first and second head portions 124, 126. Also preferably, first head portion 124 is about the same length as second head portion 126. However, the length of the first head portion 124 may be different than second head portion 126. Both body portion 122 and first and second head portions 124, 126 each preferably include countersunk holes 30, elongate top surface groove 40 extending between at least some of the holes, transverse slots 72, and side edge chamfer 28.

FIG. 12 shows another embodiment of the present invention in the form of a double Y-plate. Generally plate 130 comprises four partial elongate plate sections each similar to plate 70 combined as shown in FIG. 12. Plate 130 has a central body portion 132 which may have a substantially flat uninterrupted surface as shown. Alternatively, body portion 132 may contain fastener holes 30, a top surface groove 40, and transverse slots 72 (not shown). An adjacent first head portion 134 and a second head portion 136, combined in a manner similar to first and second head portions 124, 126 in plate 120 (see FIG. 11), are provided at either ends of body portion 132. Angle 138 is formed between first and second head portions 134, 136, respectively. Preferably, angle 138 is less than or equal to about 180 degrees. More preferably, angle 138 is about 70-110 degrees, and even more preferably about 90 degrees. In the embodiment shown, preferably first head portion 134 is about the same length as second head portion 136. However, the length of the first head portion 134 may be different than second head portion 136. First and second head portions 134, 136 also each preferably include countersunk fastener holes 30, elongate top surface groove 40 extending between at least some of the holes, transverse slots 72, and side edge chamfer 28.

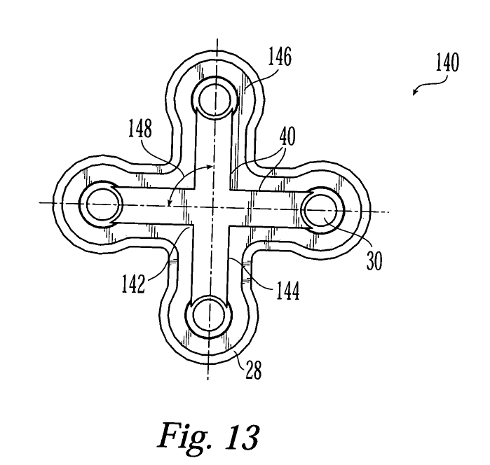

FIG. 13 shows another embodiment of the present invention in the form of an X-plate. Generally plate 140 comprises a central body portion 142 defining a common hub and four elongate arm portions 144 extending radially outward therefrom. Plate 140 may be viewed as formed by combining two intersecting elongate plate sections each similar to plate 20 and arranged generally perpendicular to each other. Each arm portion 144 is preferably disposed without limitation at an angle 148 of about 90 degrees to an adjacent arm portion 144. Each arm portion 144 also preferably includes countersunk fastener holes 30, elongate surface groove 40 extending between at least some of the holes, transverse slots 72, and side edge chamfer 28. Arm portion 144 may preferably have a slightly enlarged area 146 around countersunk hole 30 in contrast to that part of arm portion 144 connected to central body portion 142, as shown. Enlarged area 146 provides additional strength to arm portion 144 in the areas surrounding the fastener hole 30 which are typically the weakest part of a bone plate. As shown, body portion 142 may not contain any fastener holes or transverse slots, but may include elongate top surface grooves 40 as shown to provide indication of the top surface of the plate. Alternatively, body portion 142 may include transverse slots and/or fastener holes (not shown).

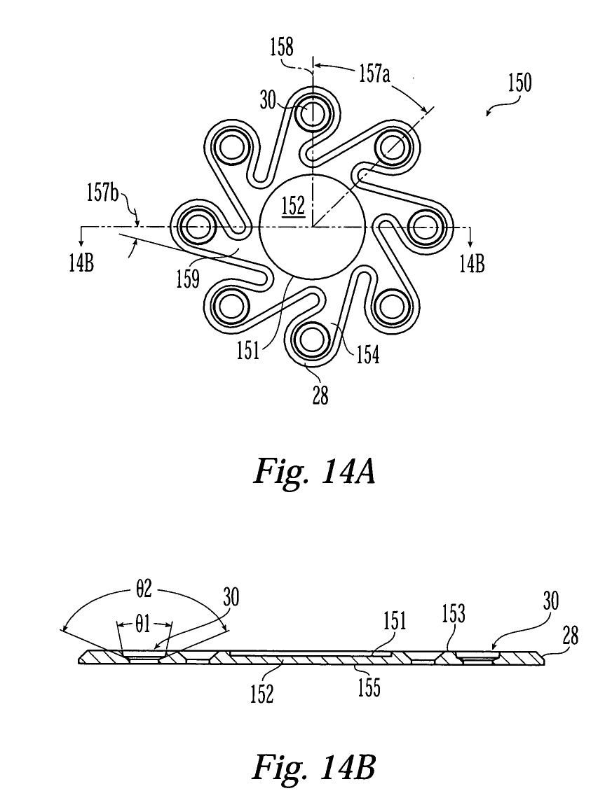

FIGS. 14A and 14B show another embodiment of the present invention in the form of a burr hole cover plate. These plates are typically used in conjunction with craniotomies wherein several spaced-apart round holes are drilled through the skull which are subsequently connected by osteotomies therebetween to create a bone flap. Generally plate 150 shown in FIG. 14A comprises a generally central body portion 152 defining a common hub and a plurality of arms 154 extending radially and angularly outward therefrom, preferably in a spiral pattern. Preferably, central body portion 152 is circular in shape. In one embodiment as shown, plate 150 preferably may have eight arms 154. However, it will be appreciated that any number of arms may be provided. The arms may extend radially outward directly in line with the plate's “radius” or the arms may extend outwardly out of line with the plate's radius, creating angle 157b as shown in FIG. 14A. It will be appreciated that arms extending out of line with the radius creates more space in between the respective arms for tool usage and therefore allows for a plate with more arms. In one embodiment, the angle 157b preferably may be about 10 to about 20 degrees, more preferably about 15 degrees, although other angles are contemplated. Each arm 154 includes a fastener hole, which preferably may be disposed on the end of each arm and may be a countersunk fastener hole 30. Fastener holes 30 may be uniformly distributed and radially spaced apart around the circumference of body portion 152. Preferably, each fastener hole 30 of an embodiment with eight arms 154 is spaced apart at angle 157a of about 45 degrees. It will be appreciated that the angle 157a between arms 154 will depend in part on the number of arms provided. As shown, each arm 154 is preferably connected to center portion 152 at a region 159 that is circumferentially offset from the radial centerline 158 of fastener hole 30 originating in the center of burr hole cover plate 150. Plate 150 also preferably includes a side edge chamfer 28.

Body portion 152 of burr hole cover plate 150 may further have a surface recess 151 that is recessed below the top surface 153 of body portion 152, and serves as a top surface indicator for plate 150. Preferably, surface recess 151 extends only partially between top surface 153 and bottom surface 155 of body portion 152 (best seen in FIG. 14B). In the embodiment shown, surface recess 151 is preferably circular in shape; however, it should be noted surface recess 151 may have any suitable shape and is not limited to circular shapes alone. In lieu of a single top surface recess 151, plate 150 may alternatively have two or more top surface recesses in any number of shapes and arranged in a variety of patterns in central body portion 152 (not shown).

Although burr hole cover plate 150 is depicted in FIG. 14A as having 8 arms 154, any suitable number of arms may be provided. Accordingly, plates 150 with fewer or more arms may be provided and are contemplated within the scope of the invention. Preferably, arms 154 are arranged in a spiral pattern similar to that depicted in FIG. 14A to facilitate fabrication of the plate by providing improved access for tooling needed to form the arms.

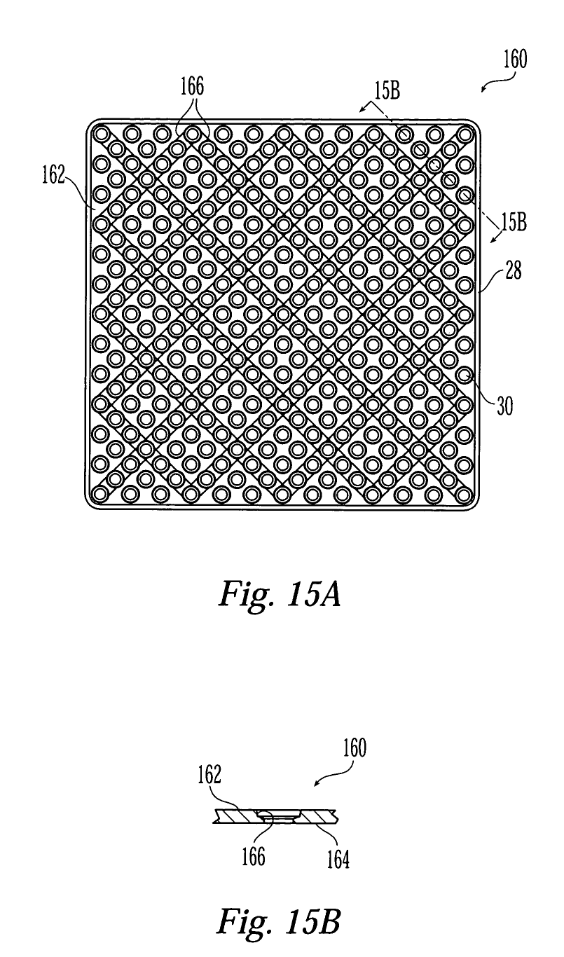

FIGS. 15A and 15B shows another embodiment of the present invention in the form of a substantially flat mesh plate. Generally mesh plate 160 comprises a top surface 162 and bottom surface 164. Optionally, mesh plate 160 may further include a side edge chamfer 28, as shown. A plurality of fastener holes 30 extend through mesh plate 160 from top surface 162 to bottom surface 164. At least one elongate top surface groove 40 extends between at least some of the holes 30. Preferably, two or more top surface grooves 40 are provided and arranged in a suitable pattern to allow the surgeon to readily detect top surface 16 of mesh plate 160. Although top surface grooves 40 are shown as preferably being oriented diagonally (with respect to the sides of mesh plate 160) and arranged in a fairly uniform pattern in the embodiment illustrated in FIG. 15A, it will be appreciated that any suitable number, orientation, or pattern may be formed with top surface grooves 40 as a matter of design choice. The invention is therefore not limited to the top surface groove configuration or pattern shown herein, and other configurations and patterns are contemplated.

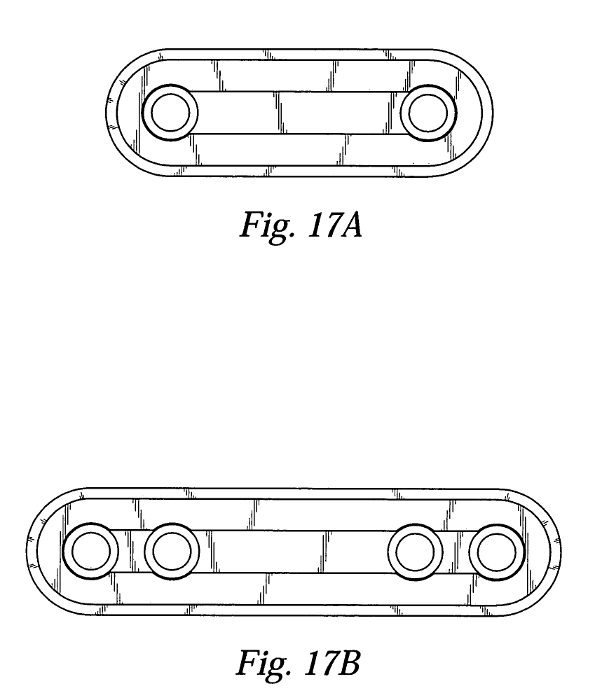

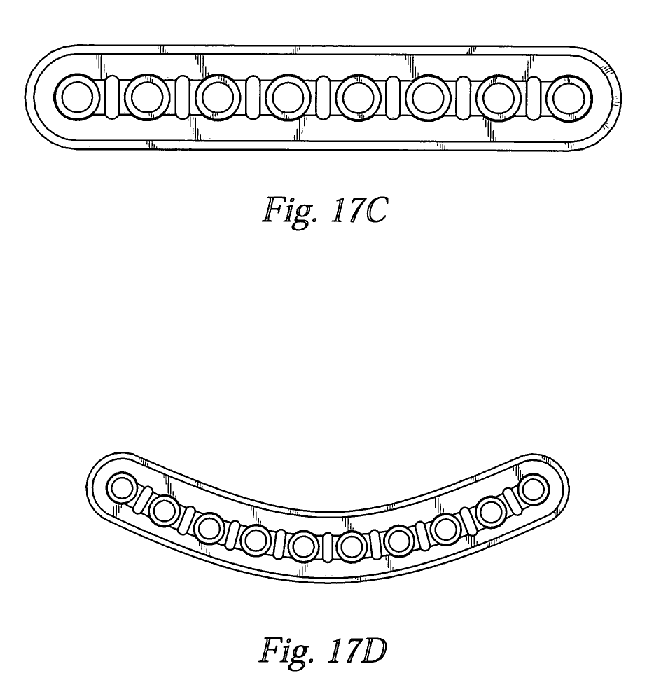

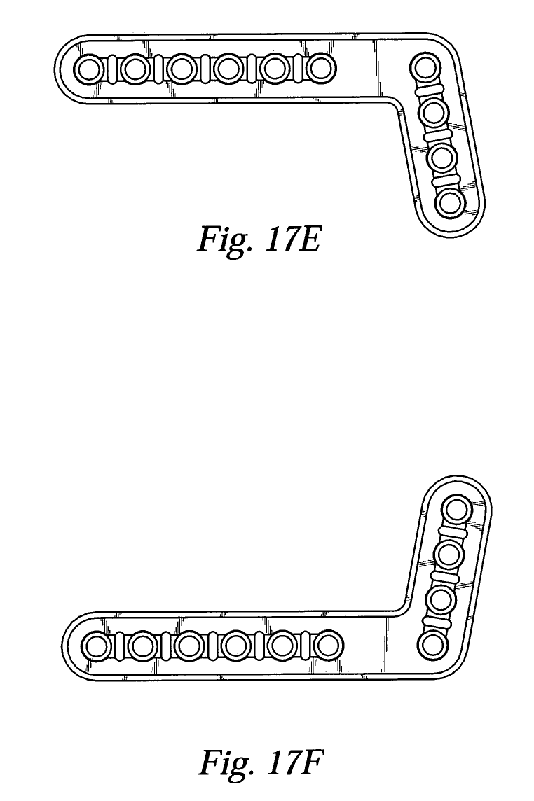

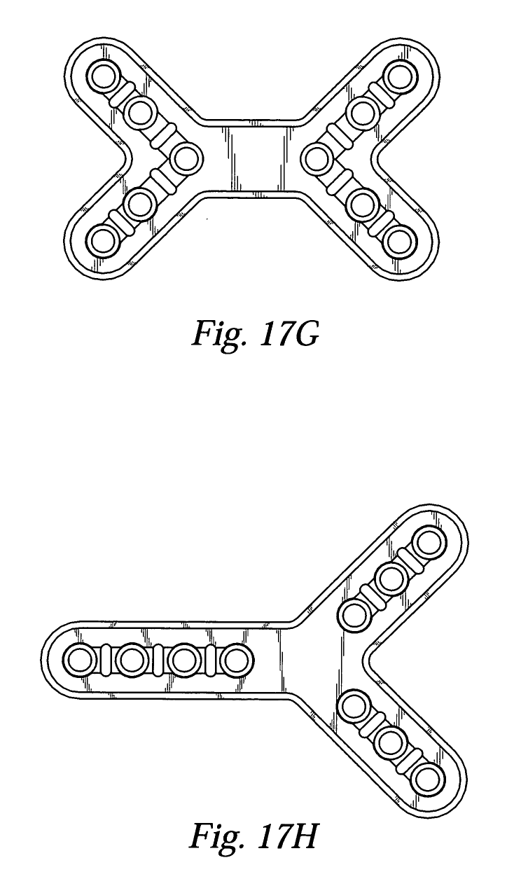

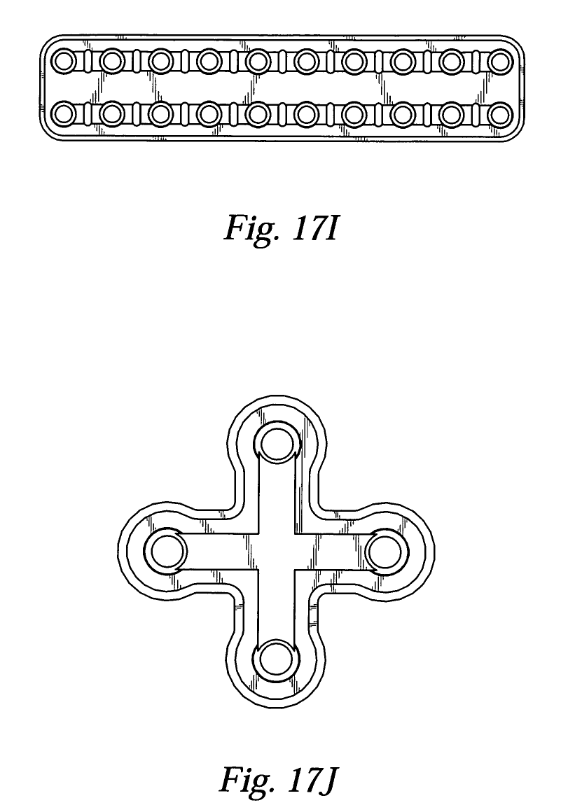

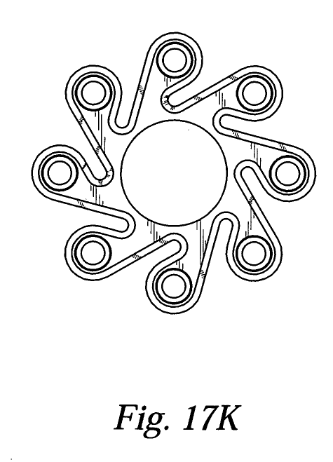

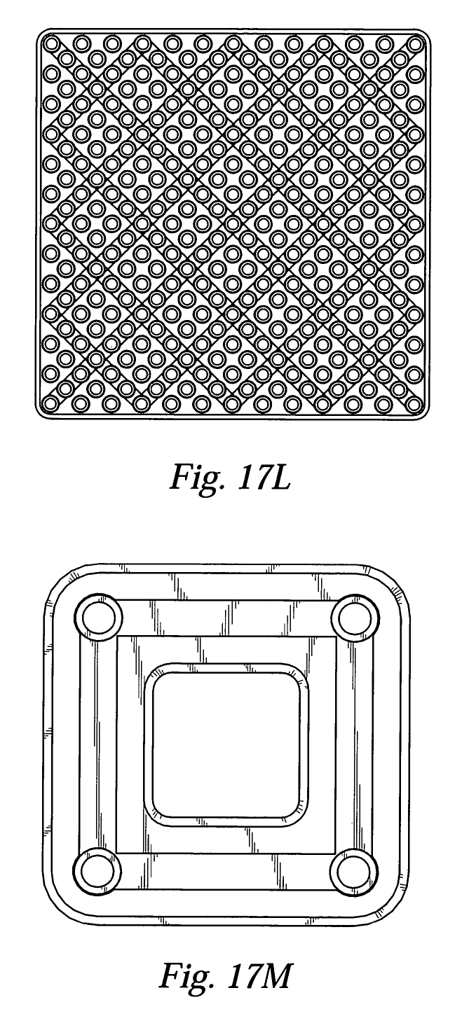

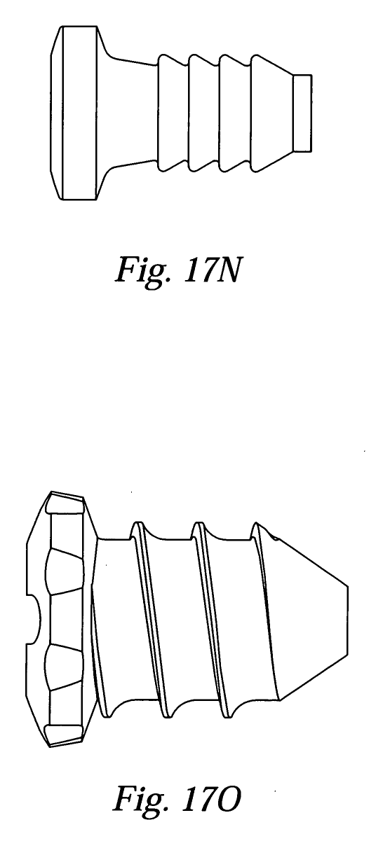

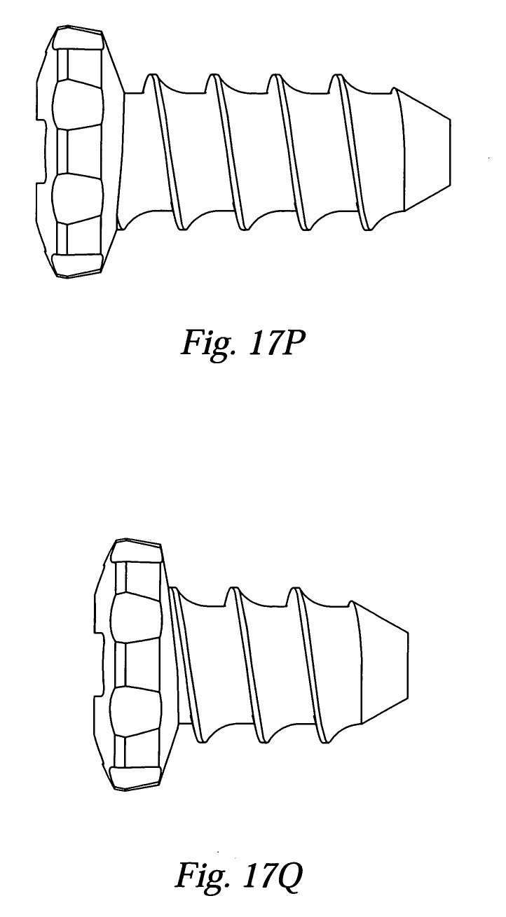

FIG. 17 shows several different components that may, in any number of combinations, compose a bone fixation kit including embodiments comprising at least a first bone implant, such as for example a bone plate, comprising a top surface and a bottom bone-contacting surface, at least two fastener holes extending from the top surface to the bottom surface, and the top surface having a recessed region that provides a tactile indicator for identifying the top surface of the implant. In one embodiment, the kit further includes a plurality of fasteners (see, e.g., FIGS. 17N-Q) for attaching the implant to a bone. In one embodiment, the top surface recess region is elongate in shape. In another embodiment, the implant includes at least one elongate plate section. The implant may have a form which includes an L-shape, a Y-shape, a double Y-shape, an X-shape, or any other style implant required for a specific procedure in another embodiment. Exemplary implant styles are shown in FIGS. 17A-M. The implant may also further comprise a length and a width, and at least one transverse slot located between the at least two fastener holes and extending across at least part of the width of the implant. Preferably, the implant is made from a resorbable material. In one embodiment, the fasteners may be screws or tacks. Exemplary screws and tacks are shown in FIGS. 17N-Q. The aforementioned embodiments are merely exemplary and, thus, this invention should not be limited to the styles or quantities shown. A kit could be custom tailored to a surgeon's preferences or to a particular procedure.

The kit may further include at least a second implant, which may be the same as the first implant, or different such as, for example, in shape, design, material, and/or dimensions including overall size (i.e., outside dimensions). The kit may also include at least a third implant the same as or different from the first and second implants. It will be appreciated that a kit according to principles of the present invention may have any number and types of implants and/or fasteners. Accordingly, numerous variations in components of the kit are possible. The kit may also include various instruments to aide in the contouring and implantation of the implant. For example, the kit may include instruments such as, but not limited to, drill bits, taps, screwdrivers, scissors, cutters, and tack drivers.

A method of contouring and implanting resorbable implants formed according to principles of the present invention will now be described with reference to FIG. 3A and plate 20 for convenience. The same method, however, applies to other embodiments formed according to principles of the present invention disclosed herein. Plate 20, preferably housed in sterile packaging and having the features described above, is provided to the surgeon in its initial rigid and flat two-dimensional form. In the surgical arena, the surgeon first determines the implant reception site on the bone and necessary final three-dimensional shape of plate 20 based on the anatomical three-dimensional shape of reception site. The surgeon next heats resorbable plate 20 to above its glass transition temperature (T_{g}) to make the plate malleable by any suitable means commonly known in the art, such as a hot water bath, hot air gun, in situ bender/cutter iron, etc., as discussed above. Preferably, the glass transition temperature (T_{g}) is above ambient operating room and human body temperatures. Preferably, the glass transition temperature (T_{g}) of the resorbable polymeric material is greater than average normal human body temperature (oral) of about 98.6 degrees Fahrenheit (37 degrees C.) so that the implant will be in a relatively rigid condition in vivo. In one embodiment, the resorbable material from which plate 20 may be made has a glass transition temperature (T_{g}) of about 131 degrees Fahrenheit (55 degrees C.) or above. The surgeon next touches the substantially planar surfaces of plate 20 to find the top surface recess 40, thereby positively identifying the top surface 22 by tactile means. Using the proper orientation with top surface 22 facing away from the bone, plate 20 may then be applied directly to the bone reception site and contoured to the desired three-dimensional shape by the surgeon. Alternatively, plate 20 may be contoured to the desired three-dimensional shape prior to being placed on the bone. A bending template can be shaped and used as an alternate means of shaping the plate. In either case, plate 20 returns to its initial rigid state as it cools below its glass transition temperature (T_{g}).

It should be noted that if the surgeon elects the alternative step noted above of shaping the heated plate before applying it to the bone, the process of heating and shaping the plate may be repeated until the surgeon is satisfied that the three-dimensional shape of the plate adequately matches the anatomical shape of the bone.

Once the surgeon is satisfied with the three-dimensional shape of plate 20, a sufficient number of holes are next drilled into the bone at various locations to preferably receive resorbable fasteners, such as without limitation bone screws 50 or tacks 160 described herein. The holes may be drilled with or without plate 20 in place on the bone. If drilled without plate 20 on the bone, plate 20 is thereafter placed and positioned onto the bone to line up the fastener holes 30 with the drilled bone-receiving holes. In either case, fasteners are then inserted through fastener holes 30 and into the pre-drilled bone-receiving holes to secure plate 20 to the bone. Since in this case fastener holes 30 are countersunk (see FIG. 3C), the surgeon is assured that the heads of either screws 50 and/or tacks 160 will be inserted through the top surface 22 of the plate and properly seated in holes 30 because the top surface 22 has been positively identified by tactile means. Thus, the top surface indictor lessens the likelihood that the plate will be improperly oriented when it is secured to the bone. It will also be appreciated that the top surface indicator also provides an additional structure which can be visually detected, as well as providing tactile identification means.

While the foregoing description and drawings represent the preferred embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description.

1. A bone implant with surface indicator, said implant comprising:
a top surface and a bottom bone-contacting surface;
at least two fastener holes extending from said top surface to said bottom surface; and
said top surface having a recessed region that provides a tactile indicator for identifying the top surface of the implant.

2. The implant of claim 1, wherein the top surface recess region is elongate in shape.

3. The implant of claim 2, wherein said implant comprises at least one elongate plate section.

4. The implant of claim 3, wherein said implant has a form selected from the group consisting of an L-shape, a Y-shape, a double Y-shape and an X-shape.

5. The implant of claim 3, wherein said implant is arcuately curved in shape.

6. The implant of claim 2, further comprising at least two top surface recess regions.

7. The implant of claim 6, wherein said implant is in the form of a substantially square mesh plate comprising four sides and at least two rows of at least two fastener holes in each of said rows, said at least two top surface recesses disposed substantially perpendicular to each other.

8. The implant of claim 7, wherein said at least two top surface recesses are disposed diagonally to said sides of said implant.

9. The implant of claim 1, wherein said top surface recess is substantially circular in shape.

10. The implant of claim 9, further comprising:
said implant having central body portion; and
a plurality of arms extending radially and angularly outward from said central body portion, said at least two fastener holes each being disposed in two different one of said arms,
whereby said implant is capable of being used as a burr hole cover.

11. The implant of claim 1, wherein said at least two fastener holes are countersunk.

12. The implant of claim 11, further comprising said countersunk holes having a first inclined wall with a first angle and an adjacent second inclined wall with a second angle, said second angle being larger than said first angle.

13. The implant of claim 12, wherein said first angle is about 20 degrees and said second angle is about 140 degrees.

14. The implant of claim 3, further comprising said implant having a length and a width, and at least one transverse slot located between said at least two fastener holes and extending across at least part of said width of said implant.

15. The implant of claim 14, wherein said transverse slot extends from said top surface to said bottom surface of said implant.

16. The implant of claim 2, wherein said recessed portion is U-shaped.

17. The implant of claim 1, further comprising at least a portion of the implant having a side edge chamfer.

18. The implant of claim 1, wherein said implant is made from a resorbable material.

19. The implant of claim 18, wherein said resorbable material is a copolymer selected from the group consisting of 70/30 poly (L, D/L-lactide) and 85/15 poly (L-lactide-co-glycolide).

20. A bone implant with surface indicator, said implant comprising:
at least one generally elongate section having longitudinal axis, a top surface, and a bottom bone-contacting surface;
at least two fastener holes in said at least one elongate section and extending from said top surface to said bottom surface; and
at least one elongate groove recessed in said top surface of said implant and extending partially between said top and bottom surfaces, said elongate groove extending at least partially between said at least two fastener holes
whereby said elongate groove provides a tactile indicator for identifying said top surface of said implant.

21. The implant of claim 20, wherein said elongate groove intersects said at least two fastener holes.

22. The implant of claim 20, wherein said elongate groove is in the form of channel.

23. The implant of claim 20, further comprising at least one elongate slot disposed in said at least one elongate section, said elongate slot disposed between said at least two fastener holes and extending transverse to the longitudinal axis of said at least one elongate section, said elongate slot further extending from said top surface to said bottom surface.

24. The implant of claim 20, wherein said implant is a substantially straight plate.

25. The implant of claim 20, wherein said implant has an arcuately curved shape.

26. The implant of claim 20, wherein said implant is made from a resorbable material.

27. A bone plate with surface indicator, said plate comprising:
a top surface and a bottom bone-contacting surface;
at least two fastener holes disposed in said plate and extending from said top surface to said bottom surface;
at least one elongate groove recessed in said top surface of said plate and extending partially between said top and bottom surfaces, said elongate groove extending at least partially between said at least two fastener holes; and
at least one elongate slot disposed in said plate and extending from said top surface to said bottom surface, said elongate slot disposed between said at least two fastener holes and extending across the plate transverse to the elongate groove, said elongate transverse slot intersecting said elongate groove;
whereby said elongate groove provides a tactile indicator for identifying said top surface of said plate; and
whereby said elongate transverse slot induces said plate to bend between the fastener holes.

28. The bone plate of claim 27, wherein said plate is made from a resorbable material.

29. The bone plate of claim 27, further comprising said plate having an elongate body portion and at least one adjacent elongate first head portion, said first head portion disposed at an angle to said body portion.

30. The implant of claim 29, further comprising said plate having a second elongate head portion disposed at an angle to said first head portion.

31. The implant of claim 27, wherein said plate has an arcuately curved shape.

32. A bone plate with bending control, said implanting comprising:
a top surface and a bottom bone-contacting surface;
at least two fastener holes disposed in said plate and extending from said top surface to said bottom surface, said two fastener holes defining a longitudinal axis therebetween; and
at least one elongate slot disposed in said plate and extending from said top surface to said bottom surface, said elongate slot disposed between said at least two fastener holes and extending transverse to said longitudinal axis;
whereby said elongate transverse slot induces said plate to bend between the fastener holes.

33. The bone plate of claim 32, wherein said plate is made from a resorbable material.

34. A method of contouring and attaching a resorbable implant with surface indicator to a bone comprising the steps of:
providing a resorbable implant having a glass transition temperature (T_{g}) that is higher than average human body temperature, said implant comprising:
a) a top surface and a bottom bone-contacting surface;
b) at least two fastener holes extending from said top surface to said bottom surface;
c) a portion of said top surface being recessed and extending partially between said top and bottom surfaces, whereby said top surface recess provides a tactile indicator for identifying said top surface of said implant;
raising the temperature of said implant to above the glass transition temperature (T_{g});
touching said surfaces of said plate to find said top surface recess thereby identifying said top surface;
deforming said plate to substantially conform to the anatomical shape of the bone with said top surface facing away from the bone;
applying said plate to the bone; and
attaching said plate to the bone.

35. The method of claim 34, further comprising the steps of:
providing fasteners;
inserting said fasteners through at least some of said fastener holes,
wherein said fasteners are used for attaching said plate to the bone.

36. The method of claim 35, wherein said fasteners are screws or tacks.

37. A bone fixation kit comprising:
at least a first bone implant comprising:
a) a top surface and a bottom bone-contacting surface;
b) at least two fastener holes extending from said top surface to said bottom surface; and
c) said top surface having a recessed region that provides a tactile indicator for identifying the top surface of the implant; and
a plurality of fasteners for attaching said implant to bone.

38. The kit of claim 37, wherein the top surface recess region is elongate in shape.

39. The kit of claim 38, wherein the implant comprises at least one elongate plate section.

40. The kit of claim 39, wherein said implant has a form selected from the group consisting of an L-shape, a Y-shape, a double Y-shape and an X-shape.

41. The kit of claim 37, further comprising said implant having a length and a width, and at least one transverse slot located between said at least two fastener holes and extending across at least part of said width of said implant.

42. The kit of claim 37, wherein said implant is made from a resorbable material.

43. The kit of claim 37, wherein said fasteners include screws or tacks.

44. The kit of claim 37, further comprising at least a second bone implant.

45. The kit of claim 44, wherein said second bone implant has a different overall size than said at least first bone implant.

46. The kit of claim 44, wherein said second bone implant has a different shape than said at least first bone implant.

47. The kit of claim 44, further comprising at least a third bone implant.

48. The kit of claim 47, wherein said third fixation device has a different shape than said at least first and second bone implants.

49. The kit of claim 47, wherein said third fixation device has a different overall size than said at least first and second bone implants.
